(19) United States
(12) Patent Application Publication
(10) Pub. No.: US 2003/0036548 A1
(43) Pub. Date: Feb. 20, 2003

(54) Method for treating anhedonia using dopamine agonists

(75) Inventors: Erich Lehr, Waldalgesheim (DE); Joachim Mierau, Mainz (DE); Michael P. Pieper, Bingen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/213,296

(22) Filed: Aug. 6, 2002

Related U.S. Application Data
(60) Provisional application No. 60/312,332, filed on Aug. 14, 2001.

(30) Foreign Application Priority Data
Aug. 10, 2001 (DE) 101 38 275.8

(51) Int. Cl. A61K 31/48

(52) U.S. Cl. 514/288

(57) ABSTRACT

A method of treating anhedonia in a patient in need thereof, comprising administering to the patient an effective amount of a dopamine agonist

### RELATED APPLICATIONS

Benefit under 35 U.S.C. § 119(e) of prior U.S. provisional application Serial No. 60/312,332, filed Aug. 14, 2001, is hereby claimed.

### FIELD OF THE INVENTION

The invention relates to the use of dopamine agonists for preparing a pharmaceutical composition for overcoming and/or alleviating anhedonia.

### BACKGROUND OF THE INVENTION

The term anhedonia is used in the prior art to denote a series of symptomatic conditions. Thus, the word anhedonia is used, for example, to denote loss of pleasure in life as well as an inability to derive any enjoyment from experiences or stimulations which normally give pleasure. Occasionally, anhedonia is divided into social anhedonia (for example, the loss of pleasure in being with friends) and psychic anhedonia (for example, the loss of pleasure in observing the beauty of nature). As a symptom anhedonia is found in psychiatric clinical pictures such as severe depression, schizophrenia, and dependency diseases. It may possibly also occur as a result of serious stress and extreme situations.

### DESCRIPTION OF THE INVENTION

It has now been found that, surprisingly, dopamine agonists may usefully be used in therapeutically effective doses to overcome and/or alleviate anhedonia.

Accordingly, the present invention relates to the use of dopamine agonists for preparing a pharmaceutical composition for overcoming and/or alleviating anhedonia.

Preferably, the present invention relates to the use of dopamine agonists for preparing a pharmaceutical composition for overcoming and/or alleviating anhedonia in diseases of dependency.

By dependency diseases are meant within the scope of the present invention diseases or disorders of the state of health, which result from the physical and/or psychological dependency of an individual on drugs and/or medication, for example. Dependency on medication may arise, for example, as a result of regularly taking active substances, such as opiates, for example. Drug dependency may arise, for example, as a result of regularly taking heroin, cocaine, marijuana, and the like. By drug dependency is also meant within the scope of the present invention physical and/or psychological dependency on alcohol, caffeine, or nicotine by the regular consumption of alcoholic or caffeine-containing drinks and tobacco products.

By dependencies for the purposes of the present invention are also meant general, non-substance-related dependencies, such as may be observed, for example, in bulimia or addiction to exercise, etc.

The withdrawal of accustomed, rewarding triggers generally leads to a number of pathological psychophysiological reactions. Therapeutic approaches are known in the prior art in which attempts are made to substitute the original addiction triggers with other, less harmful substances. These are intended to alleviate the withdrawal without themselves leading to dependency. A more targeted approach is to analyze the symptoms of the dependency more precisely and then specifically to eliminate these. Admittedly, this is only treating the symptoms to begin with, but as a result of being freed from the craving for more addiction-producing agents over and over again, the body is given the time it needs to recover in the longer term.

During withdrawal, states of excitement and restlessness as well as marked anhedonia occur in particular. Whereas attempts have already been made to treat the former with corresponding preclinical approaches, until now there have been no suitable preclinical models for specifically treating anhedonia. This is where the present invention comes in. In a newly developed experiment it has been possible preclinically for the first time to make anhedonia probable in animals, and surprisingly the substances claimed have worked convincingly in this very model. They relieve the symptoms of anhedonia with a convincing degree of reproducibility at unusually low doses. Until now, such a convincing activity has not been detected with any other substance.

Preferred dopamine agonists which may be used within the scope of the present invention are selected from among pramipexole, talipexole, ropinirole, apomorphine, lisuride, terguride, pergolide, cabergoline, bromocriptine, (−)-quinpirol, and (+)-7-OH-DPAT, optionally in the form of their enantiomers, optionally in the form of the pharmacologically acceptable acid addition salts thereof, and optionally in the form of the hydrates and solvates thereof.

Particularly preferred dopamine agonists within the scope of their use according to the invention are selected from among pramipexole, talipexole, and ropinirole, optionally in the form of their enantiomers, optionally in the form of the pharmacologically acceptable acid addition salts thereof, and optionally in the form of the hydrates and solvates thereof.

Of exceptional importance within the scope of their use according to the invention are the dopamine agonists selected from pramipexole and talipexole, optionally in the form of their enantiomers, optionally in the form of the pharmacologically acceptable acid addition salts thereof, and optionally in the form of the hydrates and solvates thereof.

Any reference to one of the abovementioned dopamine agonists includes a reference to any enantiomers of the compound in question which may exist. For example, a reference to pramipexole also includes a reference to the (+)-enantiomer as well as the (−)-enantiomer. Within the scope of the present invention, however, the (−)-enantiomer is of particular importance.

The dopamine agonists which may be used according to the invention may optionally be used in the form of the pharmaceutically acceptable acid addition salts thereof as well as optionally in the form of its hydrates and/or solvates. By pharmaceutically acceptable acid addition salts of the dopamine agonists are meant according to the invention the salts selected from the salts of hydrochloric acid, hydrobromic acid, sulfuric acid, phosphoric acid, methanesulfonic acid, acetic acid, fumaric acid, succinic acid, lactic acid, citric acid, tartaric acid, and maleic acid, of which the salts of hydrochloric acid, hydrobromic acid, sulfuric acid, phosphoric acid, and acetic acid are particularly preferred. The salts of hydrochloric acid are particularly important.

In the case of pramipexole which is particularly preferably used according to the invention, the hydrochlorides are preferably used, pramipexole dihydrochloride being of particular significance. Of the hydrates of pramipexole, pramipexole dihydrochloride monohydrate is particularly preferred.

The dopamine agonists which may be used according to the invention may optionally be used in combination with other active substances. Preferred partners in the combination are compounds selected from the categories of the antidepressants, tranquilizers and sedatives. Synergistic effects in the intended activity mean that when combinations containing one of the additional active substances mentioned above as well as the dopamine agonists are used the dosage of the individual components is reduced.

The dosage of the dopamine agonists naturally depends to a great extent on the severity of the symptoms to be treated, on the one hand, and on the choice of active substance, on the other. For example, without restricting the present invention thereto, some possible doses will now be given, particularly for the compound pramipexole which is particularly preferred according to the invention. This compound may be used in doses of about 0.05 mg to 3 mg, preferably 0.1 mg to 1.5 mg per day. These doses are based on pramipexole in the form of its free base. Based on the salt form pramipexole dihydrochloride monohydrate which is preferably used, the doses mentioned above correspond to about 0.07 mg to 4.26 mg, preferably 0.14 mg to 2.13 mg of pramipexole dihydrochloride monohydrate per day.

One possible dosing method, which is to be understood as being merely an illustrative example, is described below, based on pramipexole in the form of its free base: individual dosage titration at weekly intervals depending on activity and acceptability.

1st week: 1 tablet containing 0.088 mg of pramipexole 3 times a day;

2nd week: 1 tablet containing 0.18 mg of pramipexole 3 times a day; and

3rd week and thereafter: ½ tablet containing 0.7 mg of pramipexole 3 times a day.

Within the scope of the use according to the invention, the dopamine agonists may be administered orally, transdermally, intrathecally, by inhalation, or parenterally. Suitable preparations include, for example, tablets, capsules, suppositories, solutions, syrups, emulsions, dispersible powders, or patches. Regarding possible embodiments of a transdermal preparation which may be used according to the invention, we now refer to the embodiments described by way of example in U.S. Pat. No. 5,112,842, which is hereby incorporated by reference in its entirety. Suitable tablets may be produced, for example, by mixing the active substance or substances with known excipients, for example, inert diluents, such as calcium carbonate, calcium phosphate or lactose, disintegrants such as corn starch or alginic acid, binders such as starch or gelatine, lubricants such as magnesium stearate or talc, and/or agents for achieving delayed release such as carboxymethylcellulose, cellulose acetate phthalate, or polyvinyl acetate. The tablets may also consist of several layers.

The following are some examples of pharmaceutical preparations which may be used according to the invention. These are intended solely as an illustration without restricting the subject matter of the invention thereto.

| | |
|---|---|
| Ingredient | Amount (mg) |

Tablet 1

| | |
|---|---|
| pramipexole dihydrochloride monohydrate | 1.00 |
| mannitol | 121.50 |
| maize starch | 79.85 |
| highly dispersed silicon dioxide, anhydrous | 2.30 |
| Polyvidone K25 | 2.35 |
| magnesium stearate | 3.00 |
| Total | 210.00 |

Tablet 2

| | |
|---|---|
| pramipexole | 0.5 |
| mannitol | 122.0 |
| maize starch, dried | 61.8 |
| maize starch | 18.0 |
| highly dispersed silicon dioxide, anhydrous | 2.4 |
| Polyvidone K25 | 2.3 |
| magnesium stearate | 3.0 |
| Total | 210.0 |

Tablet 3

| | |
|---|---|
| pramipexole | 0.25 |
| mannitol | 61.00 |
| maize starch | 39.90 |
| highly dispersed silicon dioxide, anhydrous | 1.20 |
| Polyvidone K25 | 1.15 |
| magnesium stearate | 1.5 |
| Total | 105.00 |

Tablet 4

| | |
|---|---|
| pramipexole | 0.125 |
| mannitol | 49.455 |
| maize starch dried | 25.010 |
| maize starch | 7.300 |
| highly dispersed silicon dioxide, anhydrous | 0.940 |
| Polyvidone K25 | 0.940 |
| magnesium stearate | 1.230 |
| Total | 85.000 |

Solution for injection

| | |
|---|---|
| pramipexole dihydrochloride monohydrate | 0.3 mg |
| sodium chloride | 0.8 mg |
| benzalkonium chloride | 0.01 mg |
| water for injection | ad 100 ml |

We claim:

1. A method of treating anhedonia in a patient in need thereof, comprising administering to the patient an effective amount of a dopamine agonist.

2. The method of claim 1, wherein the anhedonia is associated with a dependency disease.

3. The method of claim 1, wherein the dopamine agonist is selected from pramipexole, talipexole, ropinirole, apomorphine, lisuride, terguride, pergolide, cabergoline, bromocriptine, (−)-quinpirol, and (+)-7-OH-DPAT, or an enantiomer, pharmacologically acceptable acid addition salt, hydrate, or solvate thereof.

4. The method of claim 2, wherein the dopamine agonist is selected from pramipexole, talipexole, ropinirole, apomorphine, lisuride, terguride, pergolide, cabergoline, bromocriptine, (−)-quinpirol, and (+)-7-OH-DPAT, or an enantiomer, pharmacologically acceptable acid addition salt, hydrate, or solvate thereof.

5. The method of claim 1, wherein the dopamine agonist is selected from pramipexole, talipexole, and ropinirole, or an enantiomer, pharmacologically acceptable acid addition salt, hydrate, or solvate thereof.

6. The method of claim 2, wherein the dopamine agonist is selected from pramipexole, talipexole, and ropinirole, or an enantiomer, pharmacologically acceptable acid addition salt, hydrate, or solvate thereof.

7. The method of claim 1, wherein the dopamine agonist is pramipexole, or an enantiomer, pharmacologically acceptable acid addition salt, hydrate, or solvate thereof.

8. The method of claim 2, wherein the dopamine agonist is pramipexole, or an enantiomer, pharmacologically acceptable acid addition salt, hydrate, or solvate thereof.

9. The method of claim 1, wherein the dopamine agonist is pramipexole dihydrochloride.

10. The method of claim 2, wherein the dopamine agonist is pramipexole dihydrochloride.

11. The method of claim 1, wherein the dopamine agonist is pramipexole dihydrochloride monohydrate.

12. The method of claim 2, wherein the dopamine agonist is pramipexole dihydrochloride monohydrate.
